(19) United States
(12) Patent Application Publication
(10) Pub. No.: US 2005/0271609 A1
(43) Pub. Date: Dec. 8, 2005

(54) Water-based gelling agent spray-gel and its application in personal care formulation

(75) Inventors: Lin Fei, Kendall Park, NJ (US); Ronald B. Jacoby, Holland, PA (US); Neeta Patel, Monmouth Junction, NJ (US); Suman Chopra, Dayton, NJ (US)

Correspondence Address: Rosemary Miano;COLGATE-PALMOLIVE COMPANY, 909 River Road, P.O. Box 1343, Piscataway, NJ, 08855-1343, US

(73) Assignee: Colgate-Palmolive Company

(21) Appl. No.: 10/863,545

(22) Filed: Jun. 8, 2004

(51) Int. Cl. A61K 7/32

(52) U.S. Cl. 424/65

(57) ABSTRACT

A gel composition useful in treating the underarm which contains (a) about 0.5 weight % about up to the sprayable amount of a gelling agent; (b) about 0.01 to about 10 weight % of a deodorant active or an odor reducing agent or a mixture thereof; and (c) about 70 to about 99.49 weight % water, wherein the percentages are based on the total weight of the composition.

### FIELD OF THE INVENTION

The present invention is an aqueous thixotropic sprayable gel formulation, comprised of a gelling agent, water, and one or more deodorant actives and/or odor reducing agents. Also disclosed is the use of emulsification and encapsulation of essential and optional hydrophobic ingredients in the composition. This composition is primarily designed for application as an aqueous based, pump-spray underarm deodorant gel.

### BACKGROUND OF THE INVENTION

The use of synthetic smectite clays in oral care products, personal care products, and hard surface cleaning products is known in the art. Clays are typically employed in cosmetic and personal care products as phase stabilizing agents, thickeners, and suspending agents usually in combination with additional viscosity modifying ingredients.

In addition, clays are known to possess film-forming properties useful in barrier coating applications. Synthetic sodium magnesium silicate clays are hydrophilic and therefore highly applicable in aqueous-based formulations. Gels produced by synthetic smectite clays are easily produced by low shear mixing at room temperatures. Their use however appears to be limited to formulations of low ionic strength and containing little or no cationic ingredients. Clay types employed in the present invention cannot form stable thixotropic gel formulas in the presence of high salt concentrations including antiperspirant active salts. The formation of a thixotropic gel can be achieved by the interaction of fully hydrated clay and an electrolyte, such that a high viscosity colloidal dispersion with a house of cards ordered structure is achieved. The gel once formed exhibits a low shear stress threshold that enables it to flow as a liquid under shear stress and restructure as a semi-ridged gel at rest. Gels generated via the use of synthetic smectite clays possess excellent clarity with refractive indices similar to water. The incorporation of emulsified ingredients may affect the clarity of the gel ranging from very slightly turbid to white opaque. Natural clays can be used to form gels of the present invention, however these clays when hydrated are brown, tan, or grey and therefore do not produce a visually appealing product. Clay-forming gels have excellent suspending properties and have been used extensively in the suspension of pigments in paint products. Sprayable gels as disclosed here also have the advantage of adhesion to vertical or inverted surfaces such as the axillary vault. The thixotropic character of the sprayable gel permits spraying which forms gel micro-droplets on contact with fixed surfaces. Traditional aerosol or liquid pump-sprays tend to run when applied in significant quantities to vertical surfaces.

Personal Care deodorant compositions are well known in the art. Current commercialized underarm products include aerosol, stick, roll-on, and gel forms. However, deodorant product forms have been greatly restricted to aerosols and sticks. Sprayable underarm product forms, such as aerosols, contain high levels of alcohol that cause irritation and drying of skin. Aerosol deodorants are regarded as less effective product forms due to the high volatility of ethanol and propellants when applied to skin. Deodorant sticks are also usually irritating to the skin because they often contain anionic surfactants and are formulated at pH values greater than 9.0. The use of synthetic smectite clays in the formation of gels have found limited use in personal care products. However natural clays have been widely formulated with in cosmetic and toiletry products because of their ease of use in formulating and low cost. Product formulation with synthetic smectite clays requires the proper selection of ingredients limiting interactive components that bind to monomeric clay particles resulting in weak gel structure and product instability.

### DESCRIPTION OF PRIOR ART

U.S. Pat. No. 4,087,555 presents the use of hectorite clay as a stabilizing agent in a multiple phase skin cream composition.

U.S. Pat. No. 6,136,771 discloses compositions containing high percent concentrations of antibacterial agents in hand sanitizer gel which incorporate gelling agents, including clays.

U.S. Pat. No. 4,678,593 discloses the use of smectite-type clays in the production of transparent or translucent bar soaps with improved skin conditioning performance on oily skin while providing excellent visual bar aesthetics.

U.S. Pat. No. 4,752,409 discloses the use of thixotropic clay aqueous suspensions in liquid-gel automatic dishwasher compositions.

U.S. Pat. Nos. 5,298,236; 4,863,721; 4,806,338, and 5,156,834 disclose the use of clays, particularly hydrophobically modified bentone clays in antiperspirant formulations.

U.S. Pat. No. 5,112,603 discloses compositions useful as gelling agents for aqueous systems through the combined use of smectite clays and cationic polymers.

U.S. Pat. No. 6,333,054 B1 discloses a thixotropic, non-cytotoxic, topical hydrogel that contains a proven safe and effective, broad spectrum antimicrobial agent based on a unique electrolytically derived sodium hypochloride solution.

U.S. Pat. No. 6,475,496 B1 discloses an organic based cosmetic remover composition gelled to a viscosity of 25 to 500,000 centipoise with a synthetic metal silicate gelling agent.

U.S. Pat. No. 6,203,784 B1 discloses a depilatory composition having improved rinsing properties by the inclusion of a thixotropic agent and fatty materials. The preferred thixotropic agents are smectitie clays, bentonites, and synthetic nectorite clays.

U.S. patent Publication 2002/0034486 A1 discloses leave-in hair cosmetic compositions for enhancing hair volume comprised of non-spherical microparticles, a water-soluble or water swellable polymer, and an aqueous carrier such that the combination of microparticle and polymer results in a film-forming network.

### SUMMARY OF THE INVENTION

The present invention relates to compositions for the treatment of underarm odor, more particularly to an aqueous composition containing at least one deodorant active and/or an odor reducing agent, a gelling agent and water.

The invention relates to gel composition used for the treatment of underarm odor, preferably a sprayable gel composition which comprises:

(a) about 0.5 weight % to the sprayable amount of the composition of one or a mixture of gelling agents,

(b) about 0.01 to about 10 weight % of a deodorant active and/or an odor reducing agent; and

(c) about 70 to about 99.49 weight % water.

Another embodiment of the invention is directed to compositions for the treatment of underarm odor that do not use cyclomethicones.

### DETAILED DESCRIPTION OF THE INVENTION

The present invention relates to compositions for the treatment of underarm odor. In addition, the present invention constitutes a novel product form; an aqueous-based pump-spray deodorant gel, which is non-irritating, adheres well to underarm skin, and possesses unique visual attributes. Furthermore, the present invention relates to a gel composition, which can be sprayable.

This invention is a gel deodorant composition (clear, translucent or opaque) with a rigid gel structure. The rigid gel is defined as having a viscosity greater than 20,000 cPs, and preferably greater than 100,000 cPs when measured with a Brookfield Viscometer DV-II+ at 1 rpm, 25° C. with spindle #95. Preferably the gel structure is not floatable inside a bottle under gravity, but able to be sprayed like liquid when subjected to the shear exerted by a typical trigger sprayer. A non-limiting example of such bottle is the 2 oz (56.82 ml) Cosmo Round poly(ethylene terephthalate) (“PET”) bottle (for example, the bottle made by Silgan Plastics Corporation located in Penn Yan, N.Y. with the bottle code OOM 45089). Other bottles will work. These types of formulas combine the convenience associated with spray application and elasticity from gel structure with the capability to suspend ingredients which are otherwise difficult to be stabilized in the main body. The deodorant spray gel could be a gelled solution, an emulsion/micro-emulsion, or a suspension. The gel composition preferably comprises:

(a) about 0.5 weight % to the sprayable amount of the composition, preferably from about 0.5 weight % to about 10 weight % of one or a mixture of a gelling agent such as a synthetic smectite clay material;

(b) about 0.01 to about 10 weight % a deodorant active and/or an odor reducing agent; and

(c) about 70 to about 99.49 weight % water.

The optional ingredients include:

(d) 0 to about 10 weight % gelling activator;

(e) 0 to about 5 weight % fragrances;

(f) 0 to about 10 weight % nonionic or anionic surfactant (preferably nonionic surfactant) with HLB value (hydrophilic lipophilic balance)≧12;

(g) 0 to about 25 weight %, particularly 0-15 weight %, and more particularly 0-10 weight % water soluble organic solvent (for example, ethanol, glycerol, propylene glycol, dipropylene glycol, tripropylene glycol or glycerol formal);

(h) 0 to about 20 weight %, particularly 0-10 weight % of emollient-moisturizer (for example, a member of the group consisting of hydrogenated polyiosbutene (Polyiso 250), C12-15 alkyl benzoate (FINSOLV TN), PPG-3 myristyl ether, jojoba oil or mineral oil);

(i) Other conventional optional ingredients such as dyes, preservatives, vitamins, and so on can be added into the gel composition. All the percentages are based on a total weight basis unless otherwise stated.

Water insoluble materials can be either solubilized by surfactant, or uniformly dispersed into the gel network. Oils, when dispersion technology is chosen, can be first encapsulated into beads, followed by dispersing into the matrix. For oil soluble solid, such as triclosan, pre-dissolving into hydrophobic solvent is preferred before encapsulation.

A translucent composition is defined as a composition, although allowing light to pass through, it causes the light to be scattered so that it will be impossible to see clearly objects behind the translucent composition.

An opaque composition does not allow light to pass there through.

Within the context of the present invention, if the composition is clear, the composition has an optical clarity less than approximately 50 NTU (Nephelometric Turbidity Units) at room temperature (20°-25° C.), preferably having a turbidity measurement of less than approximately 30 NTU, more preferably having a turbidity measurement of less than approximately 20 NTU. Turbidity measurements as discussed in foregoing and discussed hereinafter were made with an Orbeco-Hellige #965 Direct-Reading Turbidimeter.

Thus, according to the present invention, there are differences between transparent (clear), translucent and opaque compositions.

Gelling Agent

The term “gelling agent” as used here and hereafter refers to a compound capable of increasing the viscosity of a water-based composition, or capable of converting a water-based composition to a gel or semisolid. The gelling agent, therefore, can be organic in nature, for example, a natural gum or a synthetic polymer, or can be inorganic in nature.

The following are nonlimiting examples of gelling agents that can be used in the present invention. In particular, the following compounds, both organic and inorganic, act primarily by thickening or gelling the aqueous portion of the composition: acacia, acrylates/steareth-20 methacrylate copolymer, agar, algin, alginic acid, ammonium acrylate copolymers, ammonium alginate, ammonium chloride, ammonium sulfate, amylopectin, attapulgite, bentonite, C9-15 alcohols, calcium acetate, calcium alginate, calcium carrageenan, calcium chloride, caprylic alcohol, vinyl polymers such as cross linked acrylic acid polymers with the CTFA name Carbomer such as but not limited to carbomer 910, carbomer 934, carbomer 934P, carbomer 940, carbomer 941, carboxymethyl hydroxyethylcellulose, carboxymethyl hydroxypropyl guar, carrageenan, cellulose, cellulose gum, cetearyl alcohol, cetyl alcohol, corn starch, damar, dextrin, dibenzylidine sorbitol, ethylene dihydrogenated tallowamide, ethylene dioleamide, ethylene distearamide, gelatin, guar gum, is guar hydroxypropyltrimonium chloride, hectorite, hyaluronic acid, hydrated silica, hydroxybutyl methylcellulose, hydroxyethylcellulose, hydroxyethyl ethylcellulose, hydroxyethyl stearamide-MIPA, hydroxypropylcellulose, hydroxypropyl guar, hydroxypropyl methylcellulose, isocetyl alcohol, isostearyl alcohol, karaya gum, kelp, lauryl alcohol, locust bean gum, magnesium aluminum silicate, magnesium silicate, magnesium trisilicate, methoxy PEG-22/dodecyl glycol copolymer, methylcellulose, microcrystallinc cellulose, montmorillonite, myristyl alcohol, oat flour, oleyl alcohol, palm kernel alcohol, pectin, PEG-2M is also known as Polyox WSR.RTM. N-10, which is available from Union Carbide and as PEG-2,000; PEG-5M is also known as Polyox WSR.RTM. N-35 and Polyox WSR.RTM. N-80, both available from Union Carbide and as PEG-5,000 and Polyethylene Glycol 300,000; PEG-7M is also known as Polyox WSR.RTM. N-750 available from Union Carbide; PEG 9-M is also known as Polyox WSR.RTM. N-3333 available from Union Carbide; PEG-14M is also known as Polyox WSR.RTM. N-3000 available from Union Carbide., polyacrylic acid, polyvinyl alcohol, potassium alginate, potassium aluminum polyacrylate, potassium carrageenan, potassium chloride, potassium sulfate, potato starch, propylene glycol alginate, sodium acrylate/vinyl alcohol copolymer, sodium carboxymethyl dextran, sodium carrageenan, sodium cellulose sulfate, sodium chloride, sodium polymethacrylate, sodium silicoaluminate, sodium sulfate, stearalkonium bentonite, stearalkonium hectorite, stearyl alcohol, tallow alcohol, TEA-hydrochloride, tragacanth gum, tridecyl alcohol, tromethamine magnesium aluminum silicate, wheat flour, wheat starch, xanthan gum, and mixtures thereof

The gelling agent is preferably a clay. The preferred clay is a synthetic smectite clay. If the gel is a sprayable gel then the clay may be used in an amount of about 0.5 weight % to the sprayable amount of the gel composition, preferably from about 0.5 weight % to about 10 weight %, particularly from about 1 weight % to about 5 weight %. If a clear gel is preferred, concentration of about 4 weight % or below is recommended for the gelling agent.

Examples of the synthetic smectite clay for personal care application include Laponite XLG and Laponite XLS, both are commercially available from Southern Clay Product, Inc. (Gonzales, Tex.). Laponite materials are synthetic layered magnesium silicates, and are marketed as granular, free flowing white powders. It hydrates and swells in distilled water to give clear and colorless colloidal dispersions of low viscosity. In the presence of gelling activator, it forms clear thixotropic gel.

Deodorant Active

The spray gel includes one or combination of antibacterial actives(s) in an amount sufficient to have a deodorizing effect. The deodorant actives are antibacterial actives. Antibacterial actives can be divided into water-soluble and non-water-soluble actives. Water-soluble actives can be incorporated directly into the aqueous base of the present composition. Hydrophobic actives must be incorporated through the use of micellization, emulsification, direct suspension or encapsulation. Powdered hydrophobic actives can be dissolved in the fragrance followed by being emulsified or encapsulated.

Hydrophilic antibacterial actives useful in the present invention include, but are not limited to: biguaides, such as: polyhexamethylene Biguanide (PHMB) (sold as Cosmocil CQ by Avecia) chlorhexadine digluconate (sold as Spectradyne G by Lonza LTD.);

Alkyl halophenols, such as: phenoxyethanol, known as Phenoxetal by Clariant,

Sodium Hydroxymethylglycinate sold as Suttocide A by ISP

Dimethyldimethylol Hydantoin (DMDM Hydantoin) sold as Glydant by Lonza;

Benzoic Esters (Parabens), such as: methyl-, propyl-, butyl-, and ethylparaben sold by Sutton Labs.

Imidazolidinyl Urea, known as Germall 115 by Sutton Labs.

methylchloroisothiazolinone/methylisothiazolinone sold as Kathon CG by Rohm & Haas.

Hydrophobic antibacterial actives useful in the present invention include, but are not limited to:

Halogenated diphenyl ethers, such as: 2,4,4′-trichloro-2′-hydroxydiphenyl ether commonly known as Triclosan (sold as Irgansan DP300 by Ciba Geigy);

Halogenated carbanilides, such as: 3,4,4′-trichlorocarbanilide (TCC) (sold as Nipaguard TCC by Clariant);

Alkyl halophenols;

parachlorometaxylenol (PCMX), known as Nipacide PX, by Clariant;

Sesquiterpenes; and

3, 7, 1 1-trimethyldodeca-2,6,10-trienol sold as Farnesol by Dragoco/H&R.

Other deodorant actives include antibacterial ingredients, by non-limiting example, include those selected from the group consisting of bacteriostatic quaternary ammonium compounds such as benzalkonium chloride, benzethonium chloride, 2-amino-2-methyl-1-propanol (AMP), cetyl-trimethylammonium bromide, cetyl pyridium chloride, lauryl pyridium chloride and methyl benzethonium chloride; triclocarbon; zinc salts such as but not limited to zinc phenolsulfonate; triethyl citrate; essential oils; and combinations thereof and the like. Quaternary ammonium compounds, such as cetyl pyridium chloride and benzalkonium chloride may be included, however they are not preferred in the present invention when anionic clays are used as the primary gelling agent, because cationic antibacterial agents exhibit reduced activity in the presence of anionic clays. The most preferred deodorant active is triclosan. The fragrance may also have antibacterial properties. Other deodorant actives are described in U.S. Pat. Nos. 6,652,842; 6,613,312 and 6,485,717 which are incorporated by reference for the teaching of deodorant actives.

Odor Reducing Agents

Odor reducing agents including, but not limited to, cyclodextrins, zeolites, carbon odor-controlling agents, sodium bicarbonates, and metal salts may be incorporated at 0% to about 10 weight %, preferably from 0.01 to 4 weight %, and more preferably from 0.01 to 2 weight % by weight in the invention. These agents are chemically inert materials which do not possess significant antibacterial activity. They reduce body odor through physical interaction (such as adsorption or binding) with volatile odor generating molecules. Cyclodextrins, as used herein, includes any of the known cyclodextrins such as unsubstituted cyclodextrins containing from six to twelve glucose units, especially alpha-cyclodextrin, beta-cyclodextrin, gamma-cyclodextrin and/or their derivatives and/or mixtures thereof. The term “uncomplexed cyclodextrin” as used herein means that the cavities within the cyclodextrin in the composition of the present invention should remain essentially unfilled prior to application to skin in order to allow the cyclodextrin to absorb various odor molecules when the composition is applied to the skin.

Preferred cyclodextrins for use in the present invention are alpha-cyclodextrin, beta-cyclodextrin, gamma-cyclodextrin and/or their derivatives. More preferred are beta cyclodextrin, hydroxypropyl alpha-cyclodextrin, hydroxypropyl beta-cyclodextrin, methylated-alpha-cyclodextrin or methylated-beta-cyclodextrin. The most preferred cyclodextrin is beta-cyclodextrin.

It is also preferable to use a mixture of cyclodextrins. Such mixtures absorb body odors more broadly by complexing with a wider range of odoriferous molecules having a wider range of molecular sizes. The complexation between cyclodextrin and odorous molecules occurs rapidly when wetted with body fluids. This is convenient for the user because the cyclodextrins, while on dry skin, will not fill their cavities with other environmental odors which would otherwise render them less efficient for absorbing body odors. More particularly, upon solubilization of the cyclodextrins by the body fluids, the isolated cavities become available to form inclusion complexes with the body odor molecules. Thus, ultimately, the availability of solubilized uncomplexed cyclodextrin is essential for an effective and efficient odor control performance

Cyclodextrins having small particle sizes aid in providing higher cyclodextrin surface availability for odor absorption and therefore are preferred. As used herein, the particle size refers to the largest dimension of the particle and to the ultimate (or primary) particles. Small particle cyclodextrins of this invention are those having a particle size of less than about 12 microns, preferably less than about 10 microns, and more preferably less than about 5 microns. A more complete description of the cyclodextrins, cyclodextrin derivatives, and cyclodextrin particle sizes useful in the present invention can be found in U.S. Pat. No. 5,429,628, Trinh et al., issued Jul. 4, 1995, which is incorporated herein by reference in its entirety especially for the description of cyclodextrin.

Zeolites may also be used in the present invention. A preferred class of zeolites are characterized as “intermediate” silicate/aluminate zeolites, particularity for use in absorbing amine-type odors. “High” zeolites are preferred for control of sulfur-containing odors, e.g., thiols, mercaptans. Zeolites, both “intermediate” and “high”, are explained more fully in U.S. Pat. No. 5,429,628, to Trinh et al., issued Jul. 4, 1995, which is incorporated herein by reference in its entirety, especially for the description of zeolite.

Carbon odor-controlling agents described in U.S. Pat. No. 5,429,628 which is incorporated by reference especially for the description of carbon odor-controlling agents, may be used in the present invention. Alkali metal carbonate and/or bicarbonate salts, such as sodium bicarbonate, potassium bicarbonate, potassium carbonate, cesium carbonate, sodium carbonate, and mixtures thereof, may be included in the present invention in order to help control acid-type odors. An example of sodium bicarbonate and its use as an underarm deodorant is found in U.S. Pat. No. 4,382,079, to Marschner, issued May 3, 1983, which is incorporated by reference herein in its entirety. Preferred salts are sodium carbonate monohydrate, potassium carbonate, sodium bicarbonate, potassium bicarbonate, and mixtures thereof.

Preferred odor control metal salts include micronized or nano-particulate zinc oxides. Zinc compounds have been used to ameliorate malodor, e.g., in mouth wash products, as disclosed in U.S. Pat. No. 4,325,939, issued Apr. 20, 1982 and U.S. Pat. No. 4,469,674, issued Sep. 4, 1983, to N. B. Shah, et al., both of which are incorporated herein by reference in their entireties, in particular for the disclosure of metal salts. Highly-ionized and water soluble zinc salts, such as zinc chloride, provide the best source of zinc ions. However, care must be taken in selecting zinc salts as well as their levels, since some may be irritants to the skin and therefore are not preferred for use in the present invention. These zinc salts aid in absorbing low molecular weight amine and sulfur-containing compounds.

Low molecular weight amines and/or low molecular weight sulfur-containing materials such as sulfide and mercaptans; are components of many types of malodors such as food odors (garlic, onion, etc.), breath odor, urine odors, and particularly body/perspiration odor. Various types of zinc oxides which have small particle sizes can be used in this invention. Small particle zinc oxides that are particularly useful are those in powder form and in dispersions with esters, oils, and silicones compatible with the present invention.

Powder forms of zinc oxide may be classified as “micronized” and may have a particle size in the range of 0.02-200 microns, especially in the range of 0.02-10 microns, and most especially 0.1-10 microns.

A non-limiting example of a useful micronized zinc oxide powder is Z-COTE® micronized zinc oxide available from BASF (Charlotte, N.C.) which has an average particle size (APS) of 0.2 microns. A silane treated hydrophobic Z-COTE® micronized zinc oxide is also available for oil phase dispersions. Another type of powder form zinc oxide is characterized as being in the “nanoparticle range” with non-limiting examples being NANOX™, nano-sized zinc oxide (untreated) from Elementis Specialties (Hightstown, N.J.). This product has an APS of about 60 nanometers with a broader range of 40-60 or 40-80 nanometers. Also useful is NanoGard® zinc oxide available from NanoPhase Technologies (Romeoville, Ill.) which is a 99.0+% pure zinc oxide powder with an APS of 60 nm.

Dispersions of small particle zinc oxide in esters, oils, and silicones are useful in the present invention. Non-limiting examples of useful small particle zinc oxide dispersions include; Nanosun™ Micro zinc oxide “E” 70, which is an ester coated dispersion containing 70% small particle zinc oxide available from Shamrock Technologies Inc. (Newark, N.J.); a high solids dispersion of 20-30 nm zinc oxide (45-50%) in cyclopentasiloxane, PEG-10 dimethicone, and dimethicone available under the trade name of FA50XZ4 from Kobo Products, Inc. (South Plainfield, N.J.); a high solids dispersion of 60 nm zinc oxide (45-50%) in C12-15 alkyl benzoate, triethoxycaprylylsilane, and castor oil phosphate available under the trade name of TNC65FZS from Kobo Products, Inc. (South Plainfield, N.J.); and a high solids dispersion of 100 nm zinc oxide (45-50%) in cyclomethicone, PEG-10 dimethicone, and dimethicone under the trade name of CM3K50LZM also available from Kobo Products, Inc. (South Plainfield, N.J.).

Additional non-limiting examples of odor reducing zinc metals salts which are useful in the invention include, water-soluble zinc salts such as, zinc pyrrolidonecarboxylate (more commonly known as zinc pidolate), zinc sulphate, zinc chloride, zinc lactate, zinc gluconate, zinc phenolsulphonate and zinc ricinoleate.

The deodorant active materials or the odor reducing agents or a mixture thereof are in the composition from about 0.01 to about 10 weight % and preferably from about 0.01 to about 4%, of the total weight of the composition. Although the invention can be practiced without a deodorant active material, it is preferable that a deodorant active material is present in an amount up to about 5 weight %. The invention can also be practiced without an odor reducing agent.

Water

Water is present in the invention in an amount from about 70 weight % to about 99.49 weight %, preferably from about 80 weight % to about 95 weight %, based on the total weight of the composition.

The Optional Ingredients

Gelling Activator

The gelling activator is polar compound. Gelling activators can be found in treated water, such as the tap water that a water company distributes to homes and businesses. The invention works with no “added” gel activator.

As the activator is introduced to the dispersion of the gelling agent such as the clay, the negative charge layer on the clay platelets' surfaces are reduced, so as to the electric repulsion between the surfaces. Meanwhile, the electric attraction between the negatively charged surfaces and positively charged edges results in the formation of house of cards structure—the skeleton of the clay gel. Typical activators include electrolytes (e.g. simple salts (such as NaCl), ionic surfactants, or organic salts), coalescing solvents (e.g. DMDM hydantoin), pigments, and inorganic powers (e.g. ZnO, glass powder). The activator is present in an amount of about 0 weight % to about 10 weight %, preferably from 0.01 weight % to about 5 weight %, particularly from about 0.01 weight % to about 2 weight %.

Hydrophobic ingredients may be incorporated into the gel system through emulsification or micellization. This requires the use of high HLB (>12) surfactant. HLB is a numeric rating system for the combined hydrophilic and hydrophobic characteristics of a surfactant molecule that contains both hydrophilic and hydrophobic moieties. The assignment of numerical values for HLB based upon chemical groupings in a molecule is given by A. W. Adamson in “Physical Chemistry of Surfactants,” 2^{nd} ed. (Interscience Publishers, N.Y. 1967), pp. 520-522. Suitable surfactants include anionic and nonionic surfactants. Because of the mildness and week interaction with the clay, nonionic surfactant is preferred.

Anionic surfactant includes a hydrophobic moiety, such as a hydrocarbon chain (straight or branched, saturated or unsaturated) including about 8 to about 30 carbon atoms, and particularly about 12 to about 20 carbon atoms, and further includes a hydrophilic moiety, such as sulfate, sulfonate, carbonate, phosphate, or carboxylate. Often, the hydrophobic carbon chain is etherified, such as with ethylene oxide or propylene oxide. Suitable anionic surfactants include, but are not limited to, compounds in the classes known as alkyl sulfates, alkyl ether sulfates, alkyl ether sulfonates, sulfate esters of an alkylphenoxy polyoxyethylene ethanol, alpha-olefin sulfonates, beta-alkyloxy alkane sulfonates, alkylarylsulfonates, alkyl monoglyceride sulfates, alkyl monoglyceride sulfonates, alkyl carbonates, alkyl ether carboxylates, fatty acids, sulfosuccinates, sarcosinates, octoxynal or nonoxynol phosphates, taurates, fatty taurides, fatty acid amido polyoxyethylene sulfates, isothionates, PPG-10 cetyl ether, PEG-8 laurate or mixtures thereof. Especially useful anionic surfactants are: sodium lauryl sulfate (such as Stepanol WA-100 from Stepan Company, Northfield, Ill.) and sodium laureth sulfate (such as Rhodapex ESB 3/A2 from Rhodia Inc., Cranbury, N.J.).

The present invention prefers using one or combination of several nonionic surfactants as emulsifying agents. Nonionic surfactants are a known class of materials in this art, giving low irritation and less sensitive to electrolyte comparing with ionic surfactants. Suitable nonionic surfactants include, but are not limited to, Polyoxyethylenated straight-chain alcohols, alkylphenol ethoxylates, polyoxyethylenated polyoxypropylene glycols, long-chain carboxylic acid esters, alkanolamides, and alkylpolyglycosides. Especially useful nonionic surfactants are: polyoxyethylenated straight-chain alcohols, such as Brij 35 (from Uniqema, New Castle, Del.), and Volpo-20 (from Croda Inc., Parsippany, N.J.); polyoxyethylenated polyoxypropylene glycols, such as Eumulgin L and Eumulgin HPS (both from Cognis Co., Cincinnati, Ohio); polyethylene glycol esters, such as PEG-8 laurate; polyoxyethylene and polyoxypropylene ethers, such as PPG-10 cetyl ether and oleth-20; and polyoxyethylenated sorbitol esters, such as Tween 80 (from Uniqema, New Castle, Del.).

Cationic surfactants should be avoided, as they are not compatible with the present composition.

Combination of surfactants can also be applied for emulsification as long as the resulting HLB is above about 12.

Another method to incorporate water insoluble materials is through encapsulation. The size of the capsules varies from about 10 μm to about 2 mm.

The encapsulation techniques for cosmetic materials are usually divided into the common (or called true) encapsulation and the matrix encapsulation. In the common encapsulation, active materials as content are surrounded with wall materials as an outer film. The wall material itself is not critical and any known material will work such as but not limited to gelatin/gum arabic, polyoxymethylene melamine, polyoxymethylene urea. On the other hand, in the matrix encapsulation, active materials are mingled with the matrix forming materials in the series.

Companies who can provide customized common capsules include BalChem Encapsulates (New Hampton, N.Y.), Lipo Chemicals Inc. (Paterson, N.J.), Hallcrest Ltd. (Glenview, Ill.), Arcade Marketing Inc. (New York, N.Y.), Ronald T. Dodge Company (Dayton, Ohio), etc.

Companies who can provide customized matrix capsules include BalChem Encapsulates, Lipo Chemicals Inc., Arcade Marketing Inc., Ronald T. Dodge Company, National Starch (Bridgewater, N.J.), etc.

The following U.S. patents are incorporated by reference for its disclosure of capsules and encapsulation: U.S. Pat. Nos. 6,613,359; 6,586,013; 6,251,478; 6,153,236; 6,013,286; 5,190,775; 5,051,305 and 5,393,533.

Water Soluble Organic Solvents

The carrier of the spray gel compositions of the present invention is predominantly water, but water soluble organic solvents also can be included to help solubilize composition ingredients that are not sufficiently soluble in water, or act as a humectant. Suitable solvents include short chain alcohols, like ethanol, propanol; polyols, like glycerol; glycols, like ethylene glycol, propylene glycol, and hexylene glycol; or mixtures thereof. The optional water soluble organic solvents should not adversely affect the ability of the composition to form rigid gel structure, and is typically present in an amount of 0% to about 25 weight %, preferably from about 0 to about 15 weight %, and most preferably from 0-10 weight % of the composition.

Emollient-Moisturizer

Optionally one or more emollients may be included. Emollients are a known class of materials in this art, imparting a soothing effect to the skin. These are ingredients which help to maintain the soft, smooth, and pliable appearance of the skin. Emollients are also known to reduce whitening on the skin and/or improve aesthetics. Examples of chemical classes from which suitable emollients can be found include:

(a) fats and oils which are the glyceryl esters of fatty acids, or triglycerides, normally found in animal and plant tissues, including those which have been hydrogenated to reduce or eliminate unsaturation. Also included are synthetically prepared esters of glycerin and fatty acids. Isolated and purified fatty acids can be esterified with glycerin to yield mono-, di-, and triglycerides. These are relatively pure fats which differ only slightly from the fats and oils found in nature. The general structure may be represented by Formula VI:

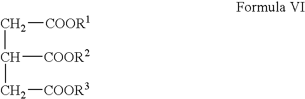

wherein each of R¹, R², and R³ may be the same or different and each have a carbon chain length (saturated or unsaturated) of about 7 to about 25. Specific examples include vegetable oil, such as peanut oil, sesame oil, avocado oil, coconut, cocoa butter, almond oil, safflower oil, corn oil, cotton seed oil, castor oil, hydrogenated castor oil, olive oil, jojoba oil, cod liver oil, palm oil, soybean oil, wheat germ oil, linseed oil, and sunflower seed oil.

(b) hydrocarbons which are a group of compounds containing only carbon and hydrogen. These are derived from petrochemicals. Their structures can vary widely and include aliphatic, alicyclic and aromatic compounds. Specific examples include paraffin, petrolatum, hydrogenated polyisobutene, and mineral oil.

(c) esters which chemically, are the covalent compounds formed between acids and alcohols. Esters can be formed from almost all acids (carboxylic and inorganic) and any alcohol. Esters here are derived from carboxylic acids and an alcohol. The general structure would be R⁴CO—OR⁵. The total number of carbons in R⁴ and R⁵ combined is in the range of about 7 to about 40 and the carbons chain can be saturated or unsaturated, straight chained or branched. Specific examples include isopropyl myristate, isopropyl palmitate, isopropyl stearate, isopropyl isostearate, butyl stearate, octyl stearate, hexyl laurate, cetyl stearate, diisopropyl adipate, isodecyl oleate, diisopropyl sebacate, isostearyl lactate, C₁₂₋₁₅ alkyl benzoates, myreth-3 myristate, dioctyl malate, neopentyl glycol diheptanoate, dipropylene glycol dibenzoate, C₁₂₋₁₅ alcohols lactate, isohexyl decanoate, isohexyl caprate, diethylene glycol dioctanoate, octyl isononanoate, isodecyl octanoate, diethylene glycol diisononanoate, isononyl isononanoate, isostearyl isostearate, behenyl behenate, C₁₂₋₁₅ alkyl fumarate, laureth-2 benzoate, propylene glycol isoceteth-3 acetate, propylene glycol ceteth-3 acetate, octyldodecyl myristate, cetyl ricinoleate, myristyl myristate.

(d) saturated and unsaturated fatty acids which are the carboxylic acids obtained by hydrolysis of animal or vegetable fats and oils. These have general structure R⁶COOH with the R⁶ group having a carbon chain length in the range of about 7 and about 25, straight chain or branched. Specific examples include lauric, myristic, palmitic, stearic, oleic, linoleic and behenic acid.

(e) saturated and unsaturated fatty alcohols (including guerbet alcohols) with general structure R⁷COH where R⁷ can be straight or branched and have about 7 to about 25 carbons. Specific examples include lauryl, myristyl, cetyl, isocetyl, stearyl, isostearyl, oleyl, ricinoleyl and erucyl alcohol.

(f) lanolin and its derivatives which are a complex esterified mixture of high molecular weight esters of (hydroxylated) fatty acids with aliphatic and alicyclic alcohols and sterols. General structures would include R⁸CH₂—(OCH₂CH₂)ₙOH where R⁸ represents the fatty groups derived from lanolin and n is form about 5 to about 75 or R⁹CO—(OCH₂CH₂)ₙOH where R⁹CO— represents the fatty acids derived from lanolin and n is from about 5 to about 100. Specific examples include lanolin, lanolin oil, lanolin wax, lanolin alcohols, lanolin fatty acids, isopropyl lanolate, ethoxylated lanolin and acetylated lanolin alcohols.

(g) alkoxylated alcohols wherein the alcohol portion is selected from aliphatic alcohols having about 2 to about 18 and more particularly about 4 to about 18 carbons, and the alkylene oxide portion is selected from the group consisting of ethylene oxide, and propylene oxide having a number of alkylene oxide units from about 2 to about 53 and, more particularly, from about 2 to about 15. Examples include cetyl glyceryl ether; isostearyl glyceryl ether; isostearyl glyceryl pentaerythrityl ether; laureth-5 butyl ether; oleyl glyceryl ether; PEG-4 ditallow ether; polyglyceryl-3 cetyl ether; polyglyceryl-4 lauryl ether; PPG-9 diglyceryl ether; propylene glycol myristyl ether. More specific examples include PPG-14 butyl ether, PPG-53 butyl ether laureth-5 butyl ether and PEG-4 ditallow ether.

(h) ethers selected from the group consisting of dicaprylyl ether; dicetyl ether; dimethyl ether; distearyl ether; ethyl ether; isopropyl hydroxycetyl ether; methyl hexyl ether; polyvinyl methyl ether.

(i) silicones as the linear organo-substituted polysiloxanes or cyclic organopolysiloxanes. The linear organo-substituted polysiloxanes are polymers of silicon/oxygen with general structure:

(1) (R¹⁰)₃SiO(Si(R¹¹)₂O)ₓSi(R¹²)₃ where R¹⁰, R¹¹ and R¹² can be the same or different and are each independently selected from the group consisting of phenyl and C1-C60 alkyl; or

(2) HO(R¹⁴)₂SiO(Si(R¹⁵)₂O)ₓSi(R¹⁶)₂OH, where R¹⁴, R¹⁵ and R¹⁶ can be the same or different and are each independently selected from the group consisting of phenyl and C₁-C₆₀ alkyl; (with specific examples including dimethicone, dimethiconol behenate, C₃₀₋₄₅ alkyl methicone, stearoxytrimethylsilane, phenyl trimethicone and stearyl dimethicone). Examples of linear silicone oils include the polydimethylsiloxanes containing from about 3 to about 9 silicon atoms. The linear volatile silicones generally have viscosities of less than about 5 centistokes at 25° C., while the cyclic materials have viscosities of less than about 10 centistokes.

The cyclic silicones include polydimethylsiloxanes containing from about 3 to about 9 silicon atoms, preferably containing from about 4 to about 7 silicon atoms, generally known as cyclomethicones. The cyclic volatile siloxanes are preferably either D4, D5 or D6, and mixtures thereof.

Examples of silicones useful in the present invention include: Dow Corning 244, Dow Corning 245, Dow Corning 344, Dow Corning 345, and Dow Corning 200 (manufactured by the Dow Corning Corporation); Silicone 7207 and Silicone 7158 (manufactured by the Union Carbide Corporation); SF1202 and SF-1173 (manufactured by General Electric).

(j) adipic acid blends selected from the group consisting of trimethyl pentanediol/adipic acid copolymer (LEXOREZ TL8 from Inolex, Philadelphia, Pa.); trimethyl pentanediol/adipic acid/isononanoic acid copolymer (LEXOREZ TC8); and adipic acid/diethylene glycol/glycerin crosspolymer (LEXOREZ 100).

(k) mixtures and blends of two or more of the foregoing.

Particular examples of suitable emollients include members of the group consisting of Octyloxyglyderin (SENSIVA SC50 from Schüilke Mayr, Norderstedt, Germany) (which can be used as an emollient as well as an antibacterial); ethoxylated alcohols such as steareth-2, nonoxynol-2, PPG-4-Ceteth-1; ethoxylated carboxylic acids such as PEG-4 dilaurate, PEG-2 oleate; glyceryl esters such as PEG-2 castor oil, polyglyceryl-3 oleate, glyceryl stearate; sorbitan derivatives such as sorbitan oleate; PPG-3 myristyl ether (such as WITCONOL APM from Goldschmidt), a dimethiconol (such as Dow Corning® DC1501 dimethiconol), neopentyl glycol diheptanoate, isocetyl stearate, dimethicone copolyol laurate, Dow Corning 2501 cosmetic wax (dimethicone copolyol); isostearyl isostearate, isostearyl palmitate, isostearyl alcohol, PPG-5-ceteth-20, triethyl hexanoin, ethyl hexyl isostearate, glyceryl oleate, isopropyl isostearate PPG-3 myristyl ether, hydrogenated polyisobutene, C12-15 alkyl benzoate and dimethicones having a viscosity in the range of 20-10,000 centistokes.

Even more particular examples include PPG-3 myristyl ether, hydrogenated polyisobutene, C₁₂₋₁₅ alkyl benzoate and dimethicones having a viscosity in the range of 100-1000 centistokes.

The emollient or emollient mixture or blend thereof incorporated in compositions according to the present invention can, illustratively, be included in amounts of 0 to about 20 weight %, preferably from about 1 weight % to about 10 weight %, preferably from about 1 weight % to about 5 weight %, more preferably from about 3 weight % to about 5 weight %, based on the total weight of the composition.

Fragrances

The fragrance may be selected from the group consisting of any cosmetically acceptable fragrance or fragrances acceptable for topical application. The fragrance should be suitable for masking malodor, such as malodor associated with human sweat. By way of non-limiting examples, these fragrances include those comprising middle note and/or top note volatile constituents, like those selected from the group consisting of allyl amyl glycolate, dihydromyrcenol, aldehyde C-12 MNA, decanol, isobornyl acetate, LILAL® tricyclo decenyl acetate, benzyl salicylate, and the like, and combinations thereof. The fragrances can also include perfumes. U.S. Pat. Nos. 6,086,903; 5,540,853 and 5,580,851 are incorporated by reference for the disclosure of fragrances and perfumes. The fragrance can be encapsulated by any known encapsulation method.

The fragrance are preferably in an amount from 0 to about 10 weight %, preferably from about 0 to 5 weight % and more preferably from 0.3 weight % to 2 weight %.

Particular formulations of the products of the invention include the following:

2-5 weight % synthetic smectite clay (for example, Laponite XLG);

0.05-0.5 weight % triclosan;

0.5-1.0 weight % fragrance;

2-5 weight % nonionic surfactant (for example, Eumulgin L);

80-95.44 weight % water;

0.01-0.05 weight % NaCl;

0-10 weight % ethanol;

wherein the composition has a viscosity in the range above 100,000 cPs, preferably above 200,000 cPs and preferably above 250,000 cPs when measured with Brookfield Viscometer DV-II+ at 1 rpm, 25° C. with Spindle# 95.

### EXAMPLES

The following Examples are offered as illustrative of the invention and are not to be construed as limitations thereon. In the Examples and elsewhere in the description of the invention, chemical symbols and terminology have their usual and customary meanings. In the Examples as elsewhere in this application values for n, m, etc. in formulas, molecular weights and degree of ethoxylation or propoxylation are averages. Temperatures are in degrees C. unless otherwise indicated. The amounts of the components are in weight percents based on the standard described; if no other standard is described then the total weight of the composition is to be inferred. Various names of chemical components include those listed in the CTFA International Cosmetic Ingredient Dictionary (Cosmetics, Toiletry and Fragrance Association, Inc., 7^{th} ed. 1997). Viscosities are determined at a temperature in the range of 20-25 degrees C. by Brookfield Viscometer DV-II+ at 1 rpm with Spindle #95.

### Examples 1-7

For Examples 1-7, the following procedure was used with the types and amounts of ingredients specified in Table A. The sample sizes were about 100 grams. To prepare Part I, water was weighted in a beaker, and stirred at 300-400 rpm using a Lightnin Mixer Model LI003. The gelling agent, Laponite XLG was then gradually added to the water. The agitation was continued for 30 minutes after the addition of laponite was finished or until the mixture became clear. Other ingredients in Part I were then added sequentially to the solution, and the resulting mixture was stirred until a clear, homogeneous solution was obtained. Ingredients in Part II were mixed in a separate beaker to form a clear solution. Part II was then added to Part I with continuous agitation, followed by the addition of Part III. After a brief agitation (about 30-60 seconds), the resulting mixture was poured into a spray bottle, and set aside at room temperature and formed a spray gel.

TABLE A

| Ingredients | 1 | 2 | 3 | 4 | 5 | 6 | 7 |
|---|---|---|---|---|---|---|---|
| Part I | | | | | | | |
| Water | 90 | 90.9 | 86.5 | 83.5 | 92.7 | 91.9 | 92.1 |
| Laponite (XLG) | 2.5 | 2.5 | 2 | 2 | 1 | 2 | 2 |
| Glycerine (99.7% | | | 5 | 8 | | | |
| CP/USP) | | | | | | | |
| Alcohol SD | | | | | | | |
| 200 proof | | | | | | | |
| CMC-7MX5 | | | | | 1 | | |
| Part II | | | | | | | |
| Eumulgin L | 5.5 | 5.5 | 4.4 | 4.4 | 4 | 5 | 5 |
| Fragrance | 1 | 1 | 0.6 | 0.6 | 0.8 | 0.6 | 0.6 |
| Triclosan | | | | | 0.2 | 0.2 | |
| Farnesol | 0.5 | | 0.5 | 0.5 | | | |
| Part III | | | | | | | |
| Glydant Liquid | 0.5 | | 1.0 | 1.0 | | | |
| (Lonza) | | | | | | | |
| NaCl (20% | | 0.1 | | | 0.3 | 0.3 | 0.3 |
| solution) | | | | | | | |
| Total | 100 | 100 | 100 | 100 | 100 | 100 | 100 |
| Viscosity* | 300 | 350 | 300 | 365 | 215 | 265 | 260 |
| (×1000 cPs) | | | | | | | |

*Viscosity was measured using a Brookfield Viscometer (Model DV-II+) with an E Spindle at 1 revolutions per minute (rpm).

### Examples 8 and 9

For Examples 8 and 9 the following procedure was used with the types and amounts of ingredients listed in Table B. The sample sizes were about 100 grams. To prepare Part I, water was weighted in a beaker, and stirred at 300-400 rpm using a Lightnin Mixer Model LI003. Laponite was then gradually added to water. The agitation was continued for 30 minutes after the addition of laponite was finished or until the mixture became clear. Part II was then added to Part I with continuous agitation. After the solution became viscous, the solution was poured into a spray bottle where Part III has been pre-added. The bottle was covered with spray pump. The bottle was then shaken several times. The beads dispersed and “locked” in the gel.

### Example 10

For Example 10 the following procedure was used with the types and amounts of ingredients listed in Table B. The sample sizes were about 100 grams. To prepare Part I, water was weighted in a beaker, and stirred at 300-400 rpm using a Lightnin Mixer Model LI003. Laponite XLG was then gradually added to water. The agitation was continued for 30 minutes after the addition of laponite was finished or until the mixture became clear. Simultaneously, in a separate beaker, ingredients in Part III were mixed and stirred until solid particles visually homogeneously dispersed. Part II was then added to Part I with continuous agitation. After the solution became viscous, Part III was added into it. The stirring speed was increased to 600 rpm. The agitation was stopped after Part III uniformly dispersed. The sample was poured into a spray bottle.

### Example 11

For Example 11 the following procedure was used with the types and amounts of ingredients listed in Table B. The sample sizes were about 100 grams. To prepare Part I, water was weighted in a beaker, and stirred at 300-400 rpm using a Lightnin Mixer Model LI003. Laponite XLG was then gradually added to water. The agitation was continued for 30 minutes after the addition of laponite was finished or until the mixture became clear. Simultaneously, in a separate beaker, ingredients in Part III were mixed and stirred until a clear solution was formed. Part II was then added to Part I with continuous agitation. After the solution became viscous, Part III was added into it. The mixture was homogenized with IKA Ultra-Turrax T25 for 5 minutes to trop down and uniformly disperse the oil droplets. The sample was poured into a spray bottle.

TABLE B

| Ingredients | 8 | 9 | 10 | 11 |
|---|---|---|---|---|
| Part I | | | | |
| Water | 89 | 93 | 86.1 | 96.5 |
| Laponite (XLG) | | 2.5 | 2.5 | 2.5 |
| Laponite (XLS) | 4 | | | |
| Part II | | | | |
| NaCl (20% w/w) | 5 | | 0.1 | 0.1 |
| Glydant Liquid (Lonza) | | 0.5 | | |
| Part III | | | | |
| Nanox (ZnO) | | | 1.0 | |
| Emulgin L | | | 0.3 | |
| Water | | | 10 | |
| Fragrance | | | | 0.8 |
| Triclosan | | | | 0.1 |
| Colored encapsulated | 2 | | | |
| beads 1* | | | | |
| Colored encapsulated | | 4 | | |
| beads 2** | | | | |
| Total | 100 | 100 | 100 | 100 |

*beads contain 50% fragrance, 45% mineral oil, and 5% triclosan.
**beads contain 80% shea butter, 18% fragrance, and 2% triclosan.

vitro Antibacterial Test

The reduction of odor-forming bacteria in the axilla is of primary importance to deodorant efficacy. In addition, many commercial underarm products contain ingredients that are irritating to skin. Therefore it was important to develop a deodorant product that would deliver an effective amount of deodorant active with little or no irritation. To demonstrate the antibacterial effectiveness of the Spray Gel an in-vitro antibacterial spray test was performed. Briefly, sterile agar plates were streaked with a standardized concentration of three representative axillary bacterial species and permitted to incubate at room temperature for 1-hour. 2 pumps (approximately 0.4 grams) of Spray Gel product was applied to the entire agar surface. The plates were incubated at 37° C. for 24-hours and then evaluated for bacterial growth. Bacterial growth was scored semi-quantitatively per plate as: 1=no visible colonies, 2=1-25 colonies, 3=26-100 colonies, and 4=>100 colonies. The samples were tested in triplicate and growth scores expressed as the average of the three plates. The results are shown below.

| | Corynebacterium | Micrococcus | Staphylococcus |
|---|---|---|---|
| Product | xerosis | luteus | epidermidis |
| Spray Gel, | 1.0 | 1.33 | 1.66 |
| Example 1 | | | |
| Spray Gel, | 4.0 | 4.0 | 4.0 |
| Example 2 | | | |
| Spray Gel, | 1.0 | 1.0 | 1.0 |
| Example 6 | | | |
| Spray Gel, | 4.0 | 4.0 | 4.0 |
| Example 7 | | | |
| Growth Controls | 4.0 | 4.0 | 4.0 |

The results demonstrate that the Spray Gel containing selected deodorant actives does deliver a bio-available active to the surface of the agar plate, in this case representing the skin surface. The negative control without deodorant actives does not inhibit bacterial growth. This demonstrates a practical application of this technology as a delivery vehicle in a deodorant spray product.

In-vivo Testing

Twenty-nine male panelists were selected to participate in a 1-week underarm deodorant efficacy study. Subjects participated in a 3-day washout period during which the use of underarm products were prohibited. At the conclusion of the washout period axillary cup-scrub extractions were performed to determine panelist's baseline axillary microflora concentrations. Panelists were then provided with 2 spray-gel formulations; one to use under each arm for a period of four days. The test products were examples are identified as #6 and #7. During the four-day product use period panelists were instructed to self assess and score axillary odor at multiple time points throughout the day after a single 0.5 gram application each morning. Axillary odor intensity was scored on a scale of 1 to 7, with 1 representing no detectable odor and 7 indicating very intense odor. At the conclusion of the four day use period a post-usage axillary cup-scrub was conducted to determine reduction in panelist's underarm microflora concentrations. An analysis of variance (ANOVA) was performed on the bacterial log reductions to test for significant differences between the two products. Differences were declared significant at the p≦0.05 level. Panelist's odor scores were compared using repeated measures analysis (RMA) with time /day, product and panelist as the effects tested. Effects were considered significant at α=0.05, with a sample size of n>24 this test was able to detect a difference in odor score of 0.6 with 80% power. The results of the in-vivo testing indicated that the experimental spray-gel sample (#6) did not provide a significant (p=0.118) reduction in total underarm microflora when compared with the control sample (#7). However, at the 12-hour and 24-hour time points on two of the four product usage days significant differences (p≦0.05) were observed in the panelist's odor intensity scores. Panelist's odor scores for time points <12-hours were not significantly different. The results therefore suggest that sufficient deodorant efficacy can be achieved particularly at longer time intervals (>12-hours) without large reductions (<0.75 Log reduction) in axillary microflora.

All the references described above are incorporated by reference in its entirety for all useful purposes. While there is shown and described certain specific structures embodying the invention, it will be manifest to those skilled in the art that various modifications and rearrangements of the parts may be made without departing from the spirit and scope of the underlying inventive concept and that the same is not limited to the particular forms herein shown and described.

1. A gel composition useful in treating the underarm which comprises:
(a) about 0.5 to about 10% of a gelling agent;
(b) about 0.01 to about 10 weight % of a deodorant active or an odor reducing agent or a mixture thereof; and
(c) about 70 to about 99.49 weight % water, wherein the percentages are based on the total weight of the composition.

2. A composition as claimed in claim 1, wherein said gelling agent is a clay.

3. A composition as claimed in claim 1, wherein said gelling agent is a synthetic smectite clay in an amount from about 1 to about 5 weight %.

4. A composition as claimed in claim 1, wherein said gelling agent is laponite.

5. A composition as claimed in claim 1, wherein the gel contains said deodorant active in an amount from 0.1 to 4% by weight.

6. A composition as claimed in claim 1, wherein the deodorant active is triclosan.

7. A composition as claimed in claim 1, wherein the gel contains said odor reducing agent.

8. A composition as claimed in claim 1, which further comprises a gelling activator.

9. A composition as claimed in claim 1, wherein said activator is present in an amount from about 0.01 weight % to about 2 weight %.

10. A composition as claimed in claim 1, wherein said water is present in an amount from about 80 to about 95 weight %.

11. A composition as claimed in claim 1, which comprises 2.0-5.0 weight % synthetic smectite clay;
0.05-0.5 weight % triclosan;
0.5-1.0 weight % fragrance;
2.0-5.0 weight % nonionic surfactant;
80-95.44 weight % water;
0.01-0.05 weight % NaCl; and
0-10 weight % ethanol.

12. A composition as claimed in claim 5, wherein said deodorant active is a water-insoluble antibacterial active.

13. A composition as claimed in claim 5, wherein said deodorant active is a water-soluble antibacterial active.

14. A composition as claimed in claim 7, wherein said odor reducing agent is a metal salt.

15. A composition as claimed in claim 7, wherein said odor reducing agent is micronized and/or nano-particulate zinc oxide, zinc ricinoleate or zinc phenolsulfonate.

16. A composition as claimed in claim 8, wherein the gelling activator is a polar compound.

17. A composition as claimed in claim 8, wherein said gelling activator is an electrolyte, an ionic surfactant, an organic salt, a coalescing solvent, a pigment, or an inorganic power.

18. A composition as claimed in claim 8, wherein said activator is present in an amount from about 0.01 weight % to about 5 weight %.

19. A composition as claimed in claim 8, wherein said gelling agent is synthetic smectite clay in an amount from about 2 to about 5 weight %, said polar compound is in an amount from about 0.01 weight % to about 2 weight %, said deodorant active is present in an amount from about 0.05 to about 5 % weight % and said water is present in an amount from about 80 to about 95 weight %.

20. A composition as claimed in claim 19, wherein said clay is laponite and said deodorant active is triclosan in an amount from 0.05 to 0.5 weight %.

21. A composition as claimed in claim 19, which further comprises at least one the following components selected from the group consisting of water soluble organic solvents, emollient moisturizers, dyes, preservatives, vitamins, and fragrances.

22. A deodorant composition comprising the composition as claimed in claim 1.

23. A sprayable gel composition used in treating the underarm which comprises:
(a) about 0.5% to the sprayable amount of the composition of a gelling agent;
(b) about 0.01 to about 10 weight % of a deodorant active or an odor reducing agent or a mixture thereof;
(c) about 0.001 to about 10 weight % of a gelling activator;
(d) about 70 to about 99.49 weight % water,
wherein the percentages are based on the total weight of the composition.

24. A sprayable composition as claimed in claim 23, wherein said gelling agent is synthetic smectite clay in an amount from about 2 to about 5 weight %, said polar compound is in an amount from about 0.01 weight % to about 2 weight %, said deodorant active is present in an amount from about 0.05 to about 5% weight% and said water is present in an amount from about 80 to about 95 weight %.

25. A composition as claimed in claim 24, wherein clay is laponite and said deodorant active is triclosan in an amount from 0.05 to 0.5 weight %.

26. A sprayable deodorant which comprises a spray bottle container containing the composition as claimed in claim 23.

27. A sprayable deodorant which comprises a spray bottle container containing the composition as claimed in claim 24.

28. A sprayable deodorant which comprises a spray bottle container containing the composition as claimed in claim 25.

29. A composition as claimed in claim 1, wherein the composition has a viscosity in the range above 100,000 cPs, when measured with Brookfield Viscometer DV-II+ at 1 rpm, 25° C. with Spindle# 95.

30. A composition as claimed in claim 1, wherein the composition has a viscosity in the range above 200,000 cPs when measured with Brookfield Viscometer DV-II+ at 1 rpm, 25° C. with Spindle# 95.

31. A composition as claimed in claim 1, wherein the composition has a viscosity in the range above 250,000 cPs when measured with Brookfield Viscometer DV-II+ at 1 rpm, 25° C. with Spindle# 95.

32. A composition as claimed in claim 1, wherein the composition has no added cyclomethicone.
